(19) United States
(12) Patent Application Publication
(10) Pub. No.: US 2026/0306868 A1
(43) Pub. Date: Oct. 8, 2026

(54) Apparatus, System and Method Using Magnetic Nerve Modulation for Treatment of Reduced Cardiac Output in the Human and Methods Related Thereto

(71) Applicant: Sonostics, Inc., Endicott, NY (US)

(72) Inventor: Kenneth McLeod, Endicott, NY (US)

(21) Appl. No.: 19/485,954

(22) PCT Filed: Jun. 11, 2024
(86) PCT No.: PCT/US2024/033335

§ 371 Date: Nov. 19, 2025

Related U.S. Application Data
(60) Provisional application No. 63/628,007, filed on Jun. 13, 2023.

(51) Int. Cl. A61N 2/02; A61N 2/00

(52) U.S. Cl. CPC A61N 2/02; A61N 2/006

(57) ABSTRACT

An apparatus is provided for a non-invasive medical intervention for a user with reduced cardiac output. The medical device includes at least one electrical coil and power control circuitry. The power control circuitry is configured to provide an electrical output supplied to the at least one electrical coil at a voltage and frequency selected to drive current through the at least one electrical coil to generate a time varying magnetic field having a frequency and an intensity that triggers a response that activates a skeletal muscle to increase a venous return of blood and a lymphatic return of interstitial fluid to improve a resting cardiac output of the user.

### BACKGROUND OF INVENTION

### 1. Field of Invention

This invention relates generally to medical intervention to improve resting cardiac output in the human. More specifically, at least one embodiment, relates to an apparatus that generates a time varying magnetic field to modulate nerve activity to trigger a response that increases the return of blood from the lower portions of the body to the heart.

### 2. Discussion of Related Art

Sedentary activity is associated with reduced levels of cardiac output in all individuals, but this decrease can be particularly large for older adults where cardiac output can commonly fall to levels which do not allow the individual to maintain normal blood pressure—this condition is referred to as “postural hypotension” and can create significant acute health challenges. More importantly, sustained low cardiac output results in reduced blood flow to all the tissues of the body resulting in reduced metabolic activity in those tissues, and correspondingly, has been shown to result in increases in cardiac events, increased risk of dementia, and increased risk of all-cause mortality.

The preferred intervention for increasing cardiac output is increased physical exercise. Exercise increases cardiac output in response to the demands of muscle activity, and as a direct result, both blood pressure and blood flow throughout the body. Increased blood flow permits an increase in the metabolic rate of the tissues throughout the body through the enhanced delivery of both oxygen and nutrients. However, large segments of modern society lead sedentary lifestyles that result in diminished cardiac output and related health complications. The health complications are numerous and can include the risk of cardio-vascular disease, the risk of developing diabetes, the risk of developing osteoporosis, and a decreased lifespan, as some examples. The United States Public Health Service has determined that, for adults, a minimum exercise level of thirty minutes per day of moderate activity is sufficient to overcome the negative impact of sedentary behavior.

The challenge with implementing exercise strategies as a preventative health measure is that most Americans cannot, or will not, follow exercise recommendations in the absence of close supervision. Numerous factors account for this reticence, including that: many people do not like to sweat; many find it difficult to fit an exercise regimen into their daily routine; many older adults are concerned about falling and injuring themselves. More specifically, walking outdoors during the winter months in the northern part of the country, or during the summer months in the southern part of the country, are not realistic expectations for many elderly people. While exercise equipment can be used in the home, such equipment can be expensive and occupies a large space within the home or apartment. In addition, many find exercise equipment confusing to operate, or are concerned about repetitive motion injury of the joints associated with continued use of such equipment.

The cardiac muscle is capable of circulating an adequate amount of blood (approximately 5-7 liters/minute for a young healthy adult) throughout the body when a person is supine (e.g. lying down). However, when a person is in an upright position (sitting or standing), there is insufficient residual pressure in the venous system to return blood from the lower parts of the body, back to the heart (a vertical distance of 1-1.5 meters) and in the absence of adequate venous return, cardiac output falls. Venous pressure, in the supine position is typically only about 20 mmHg, while a pressure greater than 100 mmHg is necessary to pump blood from the feet back to the heart against gravitational forces when a person is upright. Adequate circulation, therefore, is critically dependent on a process called “skeletal muscle pumping” to move blood back up to the heart in the upright person. While all muscles play a role in pumping blood during body motion (i.e., during breathing and exercise), when an individual is sitting or standing quietly, the muscles which play the dominant role in skeletal muscle pumping are the soleus muscles. These are specialized, deep postural muscles located in the lower legs which are responsible for returning over 70% of the blood from the lower body back to the heart when an individual is at rest in an upright posture. The soleus muscles play such a critical role in ensuring venous return that they are commonly referred to as our “second hearts.”

In the absence of adequate second heart activity, gravity will cause both blood and interstitial fluid (fluid which has extravasated from the capillaries into the surrounding tissue) to pool into the lower body (the portion of the body below the heart) whenever a person is sitting or standing quietly. Inadequate second heart activity results in health complications through three different mechanisms. First, are complications directly arising from the pooling of fluid in the lower limbs. Commonly observed symptoms of excessive lower limb fluid pooling include swollen ankles/feet/legs, lower limb joint pain, night-time leg cramps, varicose veins (leading to venous insufficiency and deep vein thrombosis), sleep apnea, as well as osteoporosis.

Second, the pooling of fluid in the lower limbs, and the resulting reduced fluid return to the heart, leads to reduced resting cardiac output. Reduced resting cardiac output, in turn, results in decreased blood flow to all the tissues of the body, limiting oxygen and nutrient flow, and therefore decreased metabolic activity in the tissues. Common symptoms of low cardiac output, due to excessive fluid pooling, include chronically cold hands and feet, peripheral neuropathy, chronic fatigue, slow wound healing, digestive disorders, and the inability to manage body weight due to reduced resting metabolic activity. Indeed, these signs and symptoms are well established indicators of second heart insufficiency in individuals who do not have a diagnosis of cardiac failure.

The third category of health complication arising from inadequate second heart activity is the development of chronic low blood pressure (postural hypotension). This condition arises when vasoconstriction is not sufficient to compensate for the drop in cardiac output and is most common in older individuals. A resting diastolic blood pressure (DBP) in the range of 80-90 mmHg is associated with the lowest all-cause mortality rates. Yet, Applicant's own study of the distribution of resting diastolic blood pressures among a sample of older (55-90 year old) men and women, found that more than 75% of older individuals have a resting diastolic blood pressure below 80 mmHg. In this study, the resting diastolic blood pressures were obtained after the individuals were seated quietly for at least 10 minutes.

A low diastolic blood pressure level is also tied to a low cognitive performance. For example, using the FDA approved Cognivue Cognitive Assessment system, a cognitive performance score below 75 is considered to be in the mild cognitive impairment range, while a score below 50 indicates moderate or severe cognitive impairment. A study of cognitive performance as a function of resting diastolic blood pressure on a selected population (individuals over age 60) identified that low cognitive performance is significantly correlated to low diastolic blood pressure levels (p=0.004). This study found that in this group of subjects the average cognitive performance score falls below 75 for diastolic blood pressure values less than 80 mmHg.

While gravity influences blood circulation in all animals, blood flow in humans is particularly affected by gravity for three reasons. First, we are relatively tall animals, and we maintain an upright posture for most of the day. Second, our skin is quite compliant. While there are other tall animals (e.g. giraffes, ostriches, horses) the skin on the legs of these upright animals is quite taut, and so does not expand easily. As a result, fluid cannot pool substantially into the lower limbs of their bodies. Third, in humans, the heart is located high in the body, such that most of the blood in the human body is below the level of the heart, whereas in most animals, 70% or more of the blood in the body is normally held above the heart.

For these reasons, humans have had to evolve a means for protecting ourselves from the forces of gravity on our blood supply. Specifically, humans require a means for pumping blood back up from the lower body to the heart when we are upright. The soleus muscles have evolved to perform this task. The soleus muscles in humans have become specialized muscles which contain multiple sinuses which can store large volumes of blood as well as interstitial fluid. When the soleus muscles contract, sufficient pressure is created within the muscle (more than 200 mmHg) to drive these sequestered fluids back up to the heart (blood is returned through the veins, interstitial fluid through the lymphatic system).

Studies illustrate the effect of soleus muscle activity when an individual transitions to an orthostatic (quiet sitting) posture from a mobile (walking) state. For example, even in healthy individuals, cardiac output during quiet sitting can fall by up to 25%-30% over the first 5-10 minutes, in some individuals, as fluids (blood and interstitial fluid) pool into the lower limbs. A subsequent increase in cardiac output usually occurs as the soleus muscles are then activated and begin to pump the pooled fluid back to the heart permitting the maintenance of normal resting cardiac output (blood flow at about 4-6 L/min).

Conversely, individuals who have inadequate soleus muscle activity experience sustained pooling when in the seated or quiet standing position, and as a result, their cardiac output will continue to fall over an extended duration. For example, in a sample of 25 women aged 45-85 years cardiac output was measured during quiet sitting (resting cardiac output). Sustained pooling was reflected in the decline in cardiac output which continued for over 90 minutes after the individuals assumed a seated posture. Cardiac index is cardiac output adjusted for body size (cardiac output divided by body surface area) and permits the averaging of cardiac output data over a group of individuals. The average drop in cardiac output for this study group was more than 35% during the 90-minute study period. Longer term studies have shown that seated individuals can pool fluid for over four hours, with cardiac output continuing to fall over the entire time-period.

Because the soleus muscles are postural muscles, they are activated primarily during specific postural activities. The soleus muscles are required, for example, during long duration standing activity—the swaying action of a person standing without support is due to the periodic contractions of the soleus muscles. Whenever the soleus muscles contract, the standing individual is pulled backwards, and at the same time, blood and interstitial fluid is pumped back to the heart. When the soleus muscles relax, the individual will begin to lean forward. The soleus muscles, therefore, act much like the cardiac muscle in that they undergo slow, cyclical contractions, helping to maintain upright posture, but also ensuring the circulatory system can work effectively when people are upright.

While the soleus muscles are used to maintain a standing posture, the primary evolutionary postural role of the soleus muscles is believed to be the maintenance of balance during squatting. This is because, when our knees are bent, the gastrocnemius muscles (the large voluntary muscles in the back of the calf) cannot produce significant contractile force, and so the only way to extend the foot (i.e. point the toes) is to use the soleus muscles. Squatting is the natural resting posture of humans, and extended squatting activity during the day is how our ancestors maintained their soleus muscles. Many cultures still utilize squatting as a common resting posture. However, following the development of inexpensive, mass produced, chairs, sitting became the most common resting posture for most people in the developed world. This change in behavior has contributed to the reduced use of the soleus muscle in adults in modern society.

In general, while children and young people commonly squat over the course of the day, most adults living in the modern world no longer utilize their soleus muscles sufficiently during the day to maintain these muscles in a healthy state. As a result, during quiet sitting most older individuals experience extensive fluid pooling into the lower body, resulting in the wide range of health complications described above. Health complications which are indicative of soleus muscle insufficiency include lower limb complications such as foot and ankle swelling, varicose veins, deep vein thrombosis, peripheral neuropathy, chronically cold hands and feet, and night-time leg cramps, etc. In addition, the lack of blood return to the heart limits cardiac output and so lowers the resting metabolic rate of the body making it difficult to maintain normal body mass, results in the development of chronic fatigue syndrome, and leads to poor wound healing. Finally, the corresponding decline in brain blood flow is associated with the development of cognitive impairment, Alzheimer's Disease, Parkinson's Disease, macular degeneration, hearing loss, and attention deficit disorder. A physician seeing a patient having any of the preceding conditions in the absence of a diagnosis of heart failure, would recognize that the individual is likely suffering from second heart insufficiency and could perform additional tests to confirm.

Studies have shown that 40%-50% of older adults have one or more of the above complications which are severe enough to significantly reduce their quality of life. Because soleus insufficiency is very much an age dependent condition, the majority of the elderly are unable to maintain normal levels of cardiac output during orthostasis (seating or standing quietly). One classic study, Katori (1979), showed that the standard cardiac output found in an upright 80-year-old is typically less than two-thirds that of a 20-year-old. In this study of cardiac output in a population of 20-80 year old individuals in a semi-reclined position, cardiac output was found to decline from a peak of close to 8 L/min in young adults, to less than 5 L/min by age 80. This 40% drop in cardiac output with age is further exacerbated by the effects of orthostatic stress during sustained quiet sitting or standing, which can cause cardiac output to drop a further 30-50%.

Prior approaches have employed mechanical stimulation of the soleus postural reflex arc to try and reverse second heart insufficiency through the mechanical stimulation of the soleus postural reflex arc. Meissner's Corpuscles, located on the plantar surfaces (soles) of the feet are a type of mechano-receptor which can detect displacement of the skin of the foot. Displacement (vertical or horizontal) of the skin on the frontal plantar surface stimulates the Meissner's Corpuscles initiating a reflex response causing the soleus muscles to contract. Conversely, activation of the Meissner's Corpuscles on the heel region of the foot serves to relax the soleus muscles.

Like all mechano-receptors, Meissner's Corpuscles are activated by specific mechanical stimuli applied at a selected frequency, leading to the triggering of the soleus postural reflex arc, and correspondingly, activation of the soleus muscles. However, while a mechanical stimulation strategy to activate the soleus muscles has been shown to be an effective intervention for retraining of the soleus muscles, this approach has numerous limitations that negatively impact its utility in practice. For example, mechanical devices suitable for use in activating the soleus muscles are relatively large, heavy devices that include significant moving parts. In addition, the mechanical sensation of the stimulation on the foot can be unpleasant for some people. Further, the mechanical nature of these devices also makes them relatively noisy which limits their usefulness in quiet environments, or situations where a large number of the devices would be in use simultaneously.

The use of electrical nerve stimulation devices applied to the human body is also known. However, any electrical nerve stimulation requires placing electrodes in contact with the skin to induce sufficient electrical current into the tissue to modify nerve activity. As a result, any electrical nerve stimulation intervention of the foot requires that the patient first remove their shoes and socks/stockings to effectively apply the electrical stimulation. This requirement is quite inconvenient for many people, especially the elderly. Further, sustained electrical stimulation can lead to rapid inactivation of the nerves due to repeated depolarization of the same nerve segments. Direct electrical nerve simulation also raises the risk of inappropriately high levels of exposure to the electrical output of the device in the event of circuit failure, as well as increasing the risk of the stimulating currents interacting with other biomedical devices (e.g. pacemakers, defibrillators, bone growth stimulators) which the patient may have in their body.

The use of magnetic nerve stimulation devices applied to the human body is also known. This is a much more recent field of study than electrical nerve stimulation. According to the current consensus of the scientific/engineering community, a time changing magnetic field must induce an electric field in the range of 5-25 V/m, or higher, into the target tissue to obtain consistent nerve excitation. To accomplish this type of stimulation typically requires a time changing magnetic flux in the range of 15-50 Tesla/second sustained for durations of at least 300 microseconds. These flux pulses are typically applied at a rate of 1-1000 Hz, as each stimulus results in the creation of a refractory period after which the nerve cannot be re-stimulated. As a result, prior technology developed to achieve magnetic nerve stimulation is designed to create relatively large magnetic flux densities for brief periods of time at a low stimulation rate.

However, the typical magnetic nerve stimulation devices described to date have many of the same limitations as the mechanical devices used to stimulate mechanoreceptors on the sole of the foot—they are large, expensive, and high-powered devices that do not lend themselves to low costs, convenience of use, or portability. In addition, the high voltages and electrical currents utilized in these devices raise many safety concerns which can significantly increase the time and cost of obtaining regulatory clearance to market the devices. The IEEE standards committee has established and published standards for safety levels with respect to human exposure to magnetic fields. This standards publication is entitled “IEEE Standards for Safety Levels with Respect to Human Exposure to Electric, Magnetic, and Electromagnetic Fields, 0 Hz to 300 GHz.” These standards identify the magnetic flux density levels to which a person can be safely exposed. For magnetic field exposures at frequencies over 3 KHz, these new exposure standards limit limb exposures (in a restricted environment—i.e. for individuals knowingly being exposed) to flux densities below 353 milliTesla RMS (494 milliTesla peak). However, head and torso exposure is limited to 0.615 milliTesla RMS (0.87 milliTesla peak) in a restricted environment. These limits on flux density represent the common knowledge of those of ordinary skill in the art. The limits also reflect an understanding by those of ordinary skill in the art that magnetic field exposures at frequencies over 3 KHz with a flux density less than 0.87 milliTesla peak have no meaningful impact on human physiology.

### SUMMARY OF INVENTION

Accordingly, there is a need to provide patients with an intervention that activates the soleus muscle to enhance venous and lymphatic blood return to the heart in adults during quiet sitting or standing, leading to normalization of cardiac output, and the ability to ameliorate, or reverse, the numerous symptoms associated with both lower limb fluid pooling, low cardiac output, and postural hypotension.

Embodiments of the invention provide an alternative approach to improve cardiac return to reduce complications of fluid pooling, and to improve blood flow to all the tissues of the body resulting in an increased level of metabolic activity in the tissues without exercise. These embodiments achieve the preceding while reducing, or eliminating, the risks and other objections people have with respect to traditional exercise regimens and/or traditional nerve and muscle stimulation technologies.

Applicants find that plantar nerve behavior can be effectively modulated with a low intensity, time varying magnetic field in such a way as to activate the soleus reflex arc. Time varying magnetic fields readily penetrate living tissue, as well as all non-magnetic man-made materials. As a result, in various embodiments, a time-changing magnetic field is utilized to directly modulate plantar nerve activity. These approaches are effective even if an individual is wearing shoes and socks. In addition, embodiments also deliver magnetic fields that reach deep into the tissue if necessary, so that, for example, in some embodiments an effective intervention is available from the top of the foot or the side of the foot. Further, various embodiments effectively activate the soleus reflex arc using time varying magnetic fields while bypassing the Meissner's Corpuscles and other skin surface receptors, so that there is no bothersome sensation felt by the user.

Applicant also finds that treatment that activates the soleus muscle via a time-varying magnetic field applied to the plantar region at an intensity and for a duration below those understood to provide nerve excitation can be employed to successfully increase resting cardiac output. Thus, according to some embodiments the treatments described herein modulate intrinsic nerve activity to effectively treat patients that have a reduced cardiac output. Further, according to various embodiments described herein, successful treatment is provided with application of magnetic fields at flux densities that are below the safe exposure levels established in the relevant industry standard. This highlights the significance of Applicant's findings because this standard would otherwise inform one of ordinary skill in the art that the flux density of the magnetic field included in these treatment protocols is inconsequential when applied to a human subject.

According to various embodiments, systems, apparatus and methods focus on directly modulating the plantar nerves, which lie deep in the tissue, while minimizing the intensity of the magnetic field exposure. Applicant has identified the optimal exposure site or area of stimulation to be treated for the most effective activation of the soleus reflex arc using time varying magnetic fields. Various embodiments also optimize the magnetic field frequency, magnetic field intensity, magnetic field orientation and duty cycle for application of the magnetic field utilized in magnetic neuromodulation for soleus muscle activation.

According to one aspect, an apparatus is provided for a non-invasive medical intervention for a user with reduced cardiac output. In various embodiments, the medical device includes at least one electrical coil and power control circuitry. The power control circuitry is configured to provide an electrical output supplied to the at least one electrical coil at a voltage and frequency selected to drive current through the at least one electrical coil to generate a time varying magnetic field having a frequency and an intensity that triggers a response that activates a skeletal muscle to increase a venous return of blood and a lymphatic return of interstitial fluid to improve a cardiac output of the user.

According to some embodiments, the frequency of the time varying magnetic field includes a range of 2-16 KHz, the flux density of the time varying magnetic field includes a range of 2-8 Gauss peak (0.2-0.8 milliTesla). According to further embodiments, the apparatus generates the time varying magnetic field during a period of 50-90 seconds at a duty cycle of 30%-50%.

According to some embodiments, the apparatus is employed in a method of treating a medical condition in a human subject where the method includes: positioning the at least one electrical coil adjacent a treatment area of the human subject, the treatment area including a targeted nerve; orienting the at least one electrical coil to locate the time varying magnetic field produced with the at least one electrical coil in a direction perpendicular to the targeted nerve; and operating the apparatus to generate the time varying magnetic field at the frequency and the intensity that triggers the response that activates the skeletal muscle to increase the venous return of blood and the lymphatic return of interstitial fluid to improve the resting cardiac output of the human subject.

According to some other embodiments, the apparatus is employed in a method of treating a medical condition in a human subject where the method includes: applying the time varying magnetic field to a treatment area located in a foot of the human subject; and selecting each of a frequency of the time varying magnetic field, a flux density of the time varying magnetic field, a period for application of the time varying magnetic field, and a duty cycle for the application of the time varying magnetic field to trigger a response that activates a soleus muscle to increase a venous return of blood and a lymphatic return of blood to improve a resting cardiac output of the human subject.

According to another aspect, a method of improving cardiac output is provided where the method includes employing an apparatus including at least one electrical coil. In various embodiments, the method includes positioning the at least one electrical coil adjacent a treatment area of a user, the treatment area including a targeted nerve, orienting the at least one electrical coil to locate a time varying magnetic field produced with the at least one electrical coil in a direction perpendicular to the targeted nerve, and operating the apparatus to generate the time varying magnetic field at a frequency and an intensity to trigger a response that activates a skeletal muscle to increase a venous return of blood and a lymphatic return of interstitial fluid to improve a cardiac output of the user.

In a further embodiment, this method of improving cardiac output is included in a method of treating an adverse medical condition, where the adverse medical condition is selected from a group consisting of: heart failure; resistant hypertension; cognitive impairment; delayed wound healing; macular degeneration; age related hearing loss; osteoporosis; and sleep apnea.

In still another embodiment, this method of improving cardiac output is employed in a method of increasing brain blood flow.

In yet another embodiment, this method of improving cardiac output is employed in a method of normalizing blood pressure in a patient diagnosed with chronically low blood pressure.

According to a further aspect, a method of improving cardiac output with an apparatus including at least one electrical coil is provided. In various embodiments, the method includes positioning the at least one electrical coil adjacent a surface of a treatment area of a user, the treatment area including a targeted nerve, orienting the at least one electrical coil to locate a time varying magnetic field produced with the at least one electrical coil in a direction perpendicular to the targeted nerve, and operating the apparatus to generate the time varying magnetic field at a frequency in a range of 2-16KHz and a flux density of 2-8 Gauss peak to trigger a response that activates a skeletal muscle to increase a venous return of blood and a lymphatic return of interstitial fluid to improve a cardiac output of the user. According to a further embodiments, the method includes generating the time varying magnetic field during a period of 50-90 seconds at a duty cycle of 30%-50%.

According to yet another aspect, a method of treating an individual for an adverse medical condition resulting is provided where for an adverse medical condition that, at least in part, results from a reduced cardiac output. According to some embodiments, the method includes applying a time varying magnetic field to a treatment area located in the individual's foot, and selecting each of a frequency of the time varying magnetic field, a flux density of the time varying magnetic field, a period for application of the time varying magnetic field, and a duty cycle for the application of the time varying magnetic field to trigger a response that activates a soleus muscle to increase a venous return of blood and a lymphatic return of blood to improve a cardiac output of the user.

Unless expressly stated otherwise herein, the term “cardiac output” refers to a resting cardiac output. Those of ordinary skill in the art will recognize based on the disclosure provided herein, that values of cardiac output and associated values concerning cardiac output such as stroke volume and stroke volume index recorded during periods of inactivity by a subject (for example, quiet standing, sitting or lying down) concern a resting cardiac output even where such periods immediately follow a period of physical activity by the subject. Those of ordinary skill in the art will also recognize based on the disclosure provided herein that any of the preceding values recorded during periods of exercise or during other activities such as walking, toe raisers, step-ups, etc. performed by a subject are not directed to resting cardiac output.

As used herein the term “user” refers to the subject that receives the treatment in the form of the time varying magnetic field. Accordingly, those of ordinary skill in the art will recognize based on the disclosure provided herein that while apparatus employed to provide the treatment can be operated by others, the user is the patient. Further, the embodiments illustrated and described herein permit direct self-treatment by patients using the apparatus to deliver the time varying magnetic field to the patient's treatment area(s) at a home, office or medical facility without the aid of a medical professional or the assistance of any other individual.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings

FIG. 1 illustrates a plot of stroke volume versus time including a period in which a magnetic field is applied to the feet of a subject in accordance with the one embodiment;

FIG. 2 illustrates a plot of vascular resistance versus time including a period in which a magnetic field is applied to the feet of a subject in accordance with the one embodiment;

FIG. 3 illustrates a bar graph showing a change in slope of vascular resistance resulting from application of a magnetic field to the feet of a subject at selected frequencies according to one embodiment;

FIG. 4 illustrates a plot of a change in stroke volume index versus a peak flux density of a magnetic field applied to the feet of a subject in accordance with the one embodiment;

FIG. 5 illustrates a location of nerves in a human foot when viewed from an underside of the foot;

FIG. 6 a plot of a change in stroke volume index versus a peak flux density of a magnetic field applied to the feet of a subject in accordance with another embodiment;

FIG. 7 illustrates a plot of a change in stroke volume index versus a duty cycle of a magnetic field applied to the feet of a subject in accordance with one embodiment;

FIG. 8 illustrates a plot of a change in stroke volume index versus an on-time of an application of a magnetic field applied to the feet of a subject in accordance with one embodiment;

FIG. 9 illustrates a plot of stroke volume index for an individual seated quietly with and without an application of a magnetic field to the feet of the subject in accordance with one embodiment;

FIG. 10 illustrates a plot of cardiac output for an individual standing quietly with and without an application of a magnetic field to the feet of the subject in accordance with one embodiment;

FIG. 11 illustrates electrical circuitry included in an apparatus employed to generate a magnetic field for application to the feet of a subject in accordance with one embodiment;

FIG. 12 illustrates a top view of an apparatus configured to generate a magnetic field for application to the feet of a subject in accordance with one embodiment;

FIG. 13 illustrates a cross-sectional view of the apparatus of FIG. 12 in accordance with one embodiment; and

FIG. 14 illustrates the apparatus of FIG. 12 being employed by a user in accordance with one embodiment.

### DETAILED DESCRIPTION

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

Applicant has designed, built and tested various embodiments of apparatus to deliver a controlled magnetic field to the plantar nerves. This testing has allowed the development of a treatment device that provides magnetic nerve modulation for a highly effective treatment of reduced cardiac output in the human patient. Further, the tests described herein have provided Applicant with heretofore unknown insights into optimal magnetic field orientation, frequency, intensity, duty cycle and treatment period to affect an improvement in soleus muscle activity that is sustainable for periods between those treatment periods when the device is being actively employed by the patient.

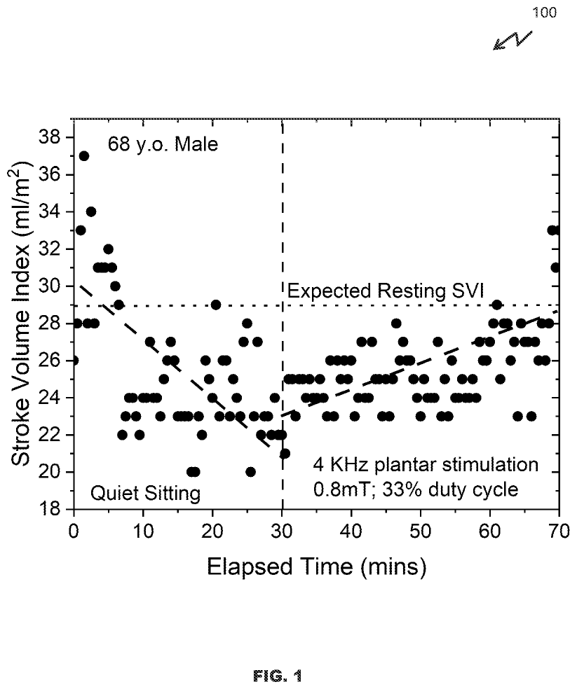

Referring now to FIG. 1, a plot 100 of stroke volume index versus time including a period in which a magnetic field is applied to the foot of a subject is illustrated in accordance with the one embodiment. FIG. 1 includes an x-axis of time in minutes and a y-axis of the stroke volume index in milliliters per square meter. The plot 100 illustrates a typical response to the neuromodulation of the plantar nerve delivered by an electro-magnetic treatment apparatus. The horizontal dashed line represents a value of the stroke volume index to which cardiac output is expected to fall to after transitioning from stand to sit in a healthy individual of the same age who is not subject to fluid pooling in the lower extremities. A drop in stroke volume index of about 20% is expected for these healthy individuals.

Here, the subject is a 68-year-old man with a known soleus insufficiency sitting quietly. The data points are measurement values of stroke volume index as recorded from the time at which the subject sits down until 70 minutes later. The solid vertical line located at the 30-minute mark represents the time when an application of the magnetic field begins. The plot demonstrates that the subject experiences a substantial and steady decline in stroke volume index while seated quietly. The data illustrates a measured decrease in stroke volume index of approximately 50% during the initial 30-minute period prior to application of a time varying magnetic field to the treatment areas.

At the 30-minute mark a time varying magnetic field is applied to the treatment areas, that is, the central regions of the subject's frontal plantar surfaces on both the right foot and left foot. In this example, a high frequency, low flux density magnetic field is applied to the treatment areas. Specifically, a sinusoidal magnetic field is provided at 4 KHz and 0.8 mT peak values. According to this embodiment, a duty cycle of 33% is employed during the treatment. The subject experiences an immediate increase in stroke volume on an initial application of the treatment. This is followed by a steady increase over the following 40 minutes achieving an approximately 45% increase in stroke volume index. This returns the subject's stroke volume to that which a healthy individual is expected to experience while sitting quietly.

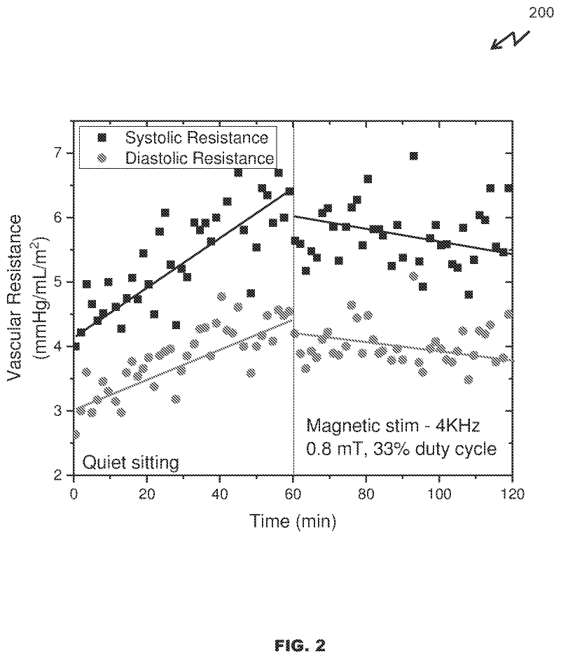

In addition to increased venous blood return, activation of the soleus muscle also acts to remove interstitial fluid from the lower limbs which in turn reduces tissue pressures in the lower limbs. A result of the combined effect of the increase in venous blood return and the removal of interstitial fluid from the lower limbs is a decrease in vascular resistance. This leads to greatly improved blood flow to the lower body. Referring now to FIG. 2, a plot 200 of vascular resistance versus time is illustrated in accordance with an embodiment in which a time varying magnetic field is applied to the foot of a subject. FIG. 2 includes an x-axis of time in minutes and a y-axis of vascular resistance in millimeters of mercury per milliliter per square meter. The data points include measured systolic resistance (represented by square-shaped points) and measured diastolic resistance (represented by circle-shaped points). Here, vascular resistance is calculated as blood pressure (BP) divided by stroke volume index. The data points are values of the respective types of vascular resistance as recorded from the time at which the subject sits down until 120 minutes later. Again, a solid vertical line located at the 60-minute mark represents the time when the treatment begins using a sinusoidal magnetic field provided at 4 KHz and 0.8 mT peak values. Here too, the illustrated embodiment is that of a magnetic field having a duty cycle of 33% being employed during treatment. The plot includes a curve fit for each of a pre-treatment change in systolic resistance (the upper of the two leftmost sloped lines) and a pre-treatment change in diastolic resistance (the lower of the two leftmost sloped lines). The plot also includes a curve fit for each of a change in systolic resistance during treatment (the upper of the two rightmost sloped lines) and a change in diastolic resistance during treatment (the lower of the two rightmost sloped lines). Each of these is representative of a rate of change and a direction of that change for the respective measurement.

The plot 200 illustrates a representative effect of magnetic field exposure of the plantar nerve on vascular resistance. During quiet sitting, blood and interstitial fluid pooling lead to both a reduction in venous return to the heart (decreased cardiac output) as well as compression of the lower limb capillaries (vasoconstriction) due to increased tissue pressure, resulting in a decline in cardiac output and a rise in blood pressure. This is reflected in a rapid increase in vascular resistance during quiet sitting. For example, the respective slopes of the systolic resistance and the diastolic resistance of the two leftmost curve fits reflect the overall increase in these values during the first 60 minutes of quiet sitting. A decline in vascular resistance begins once the application of the sinusoidal magnetic field to the treatment area begins at the 60-minute mark. This is a result of an activation of the soleus muscle which improves venous return to the heart along with removal of interstitial fluid in the lower limb tissues. The overall decrease is illustrated by the negative value of the respective slopes of the systolic resistance and the diastolic resistance illustrated in the plot 200.

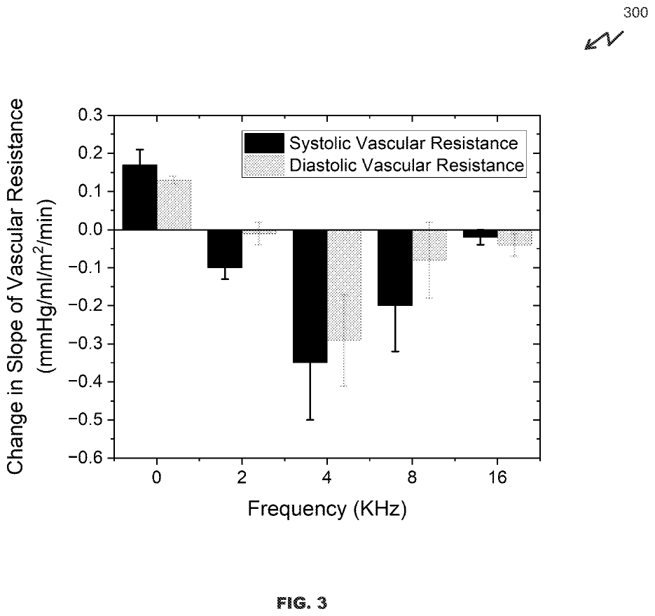

Applicant finds that the effectiveness of the treatment can be improved with a selection of a sinusoidal frequency of the time varying magnetic field at which the vascular resistance is found to decrease most rapidly relative to the control test period because this also provides the most substantial increase in cardiac output and decrease in fluid pooling over time. Referring now to FIG. 3, a bar graph 300 showing a change in slope of vascular resistance resulting from application of a magnetic field to the foot of a subject at selected frequencies is illustrated according to one embodiment. FIG. 3 includes an x-axis of frequency in hertz and a y-axis of a change in slope of vascular resistance in millimeters of mercury per milliliter per square meter per minute. The bar graph 300 includes slope values of systolic vascular resistance illustrated using bars shaded black and slope values of diastolic resistance illustrated using bars shaded gray. Here too, vascular resistance is calculated as blood pressure (either systolic or diastolic) divided by stroke volume index.

The evaluation illustrated in FIG. 3, included a determination of vascular resistance when the subject was treated with a time-varying sinusoidal magnetic field applied to an area the frontal plantar surface approximately 16 cm² in size at each of 2 KHz, 4 KHz, 8 KHz, and 16 KHz. Slope values illustrating both the rate of change of systolic vascular resistance and diastolic vascular resistance, respectively, at a control frequency of 0 KHz are also illustrated in the plot 300. These results illustrate that treatment of the plantar nerve with an application of a high frequency time varying magnetic field reduces vascular resistance. This contrasts with the control illustrated with the two leftmost bars each of which shows an increase in both systolic vascular resistance and diastolic vascular resistance.

These results also illustrate that the most effective frequency range for treatment by focal magnetic exposure of plantar nerve endings is in the range of 2 KHz to 8 KHz, with the optimal frequency being about 4 KHz. For example, the rate at which both systolic vascular resistance and diastolic vascular resistance decrease is more than twice as great with a treatment using a 4 KHz sinusoidal magnetic field than found when either 2 KHz or 8 KHz. sinusoidal magnetic field is used for treatment. Applicant finds that the effects are observed in both increased stroke volume, reflecting an increase in venous return from the lower limbs to the heart, and decreased blood pressure, resulting from the increased interstitial fluid return from the lower limbs and a corresponding decrease in tissue pressure.

In contrast to the prior understanding of those of ordinary skill in the art, Applicant evaluated the effectiveness of the treatment with a sinusoidal frequency of the time varying magnetic field greater than 3 KHz at flux densities less than 0.87 milliTesla peak. That is, Applicant evaluated treatment at flux density values considered to lack any utility by others of skill in the art. In particular, Applicant evaluated embodiments with treatment using magnetic fields that are commonly understood by those of ordinary skill in the art to be unable to influence nerve activity in the brain. These same embodiments also include treatment to a subject's feet using magnetic fields that are 1000× below levels that the relevant standards body (i.e., the IEEE) believes capable of influencing nerve activity in the limbs. However, Applicant evaluated various embodiments to determine whether flux densities well below 0.87 mT peak (i.e. 8.7 Gauss peak) would be capable of achieving effective soleus muscle activation. In addition, as described herein, Applicant evaluated the impacts of magnetic field orientation, duty cycle and on-time periods on the effectiveness of treatment.

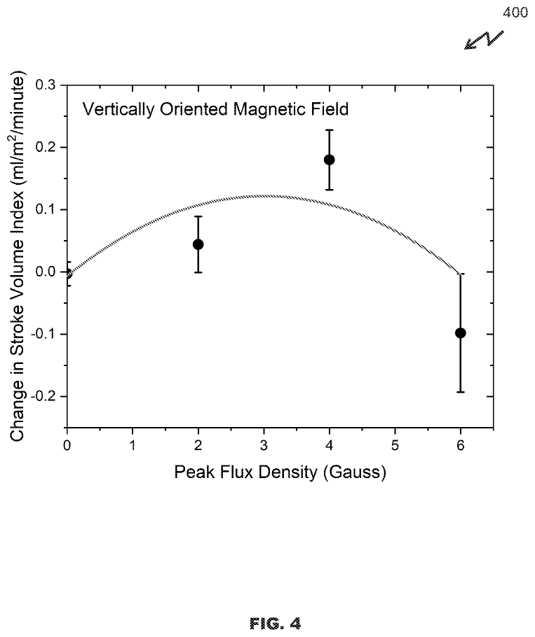

For example, Applicant employed different orientations of the electromagnets relative to the treatment area to determine the impact of changing magnetic field orientation on soleus muscle activation. Referring now to FIG. 4, a plot 400 of stroke volume index versus peak flux density is illustrated in accordance with an embodiment in which the magnetic field is oriented vertically relative to a surface on which a subject's foot is placed. FIG. 4 includes an x-axis of peak flux density in gauss and a y-axis of the stroke volume index in milliliters per square meter. A stroke volume index is used to normalize the results as determined for stroke volume across the varying body size of subjects.

For this testing, the treatment area of the magnetic nerve modulation device was increased. Initial testing according to this embodiment, the magnetic nerve modulation device employed multi-layer air-core coils of 10 cm diameter, using 90 turns of #20 magnet wire, resulting in a 1 cm thick coil, with an inductance of 1500 microHenry. These coils were driven at 4 Khz with a current sufficient to produce flux densities ranging from zero to 8 Gauss peak. Each foot of the study subjects was positioned over a coil such that only the front half (frontal plantar surface) of the foot was over the coil windings. In this position, the magnetic flux passed vertically through the foot. The standard testing protocol was utilized, with 30 minutes of quiet sitting, followed by 30 minutes of nerve modulation applied to both feet, while cardiac output was monitored. This provided the subject with exposure of the frontal plantar surface to a vertically oriented magnetic flux over a range of flux densities.

FIG. 4 illustrates the varied effectiveness of 4 Khz sinusoidal magnetic nerve modulation of the plantar nerves to achieve soleus activation with changes in flux density of the applied magnetic field. At each value of flux density, the range of values of change in stroke volume index were recorded. At each of these values of flux density, the plot 400 includes a vertical line representing the standard deviation and a single data point representing the average value recorded. The plot 400 illustrates that maximal effectiveness is observed at about 3 Gauss (0.3 mT peak) based on polynomial curve fitting. However, Applicant finds that the overall effect is relatively low. For example, this testing results in the estimated maximum effect of this vertically oriented magnetic field on stroke volume is about 0.1 ml/m² per minute.

In addition, this evaluation demonstrates that while at low flux densities, increasing magnetic nerve modulation intensity was found to be associated with increasing effectiveness, at the highest flux density tested in this series (6 Gauss peak) the effectiveness declines. This results in an apparent peak effectiveness occurring near 3 Gauss peak. With these results, Applicant identified that the non-linear response results from exposure of portions of the foot beyond the frontal plantar region being exposed to large magnetic flux densities, for example, as occurs with an increase in the magnetic flux density of the applied magnetic field. That is, portions of the mid-foot and/or heel-region of the plantar surface become exposed to large magnetic flux densities which may be significant enough to modulate nerve fibers originating in the posterior region of the foot. This is counterproductive to the treatment goals because, while activation of plantar nerve fibers in the front of the foot triggers a reflex arc leading to soleus contraction, activation of plantar nerve fibers in the posterior region of the foot lead to soleus relaxation.

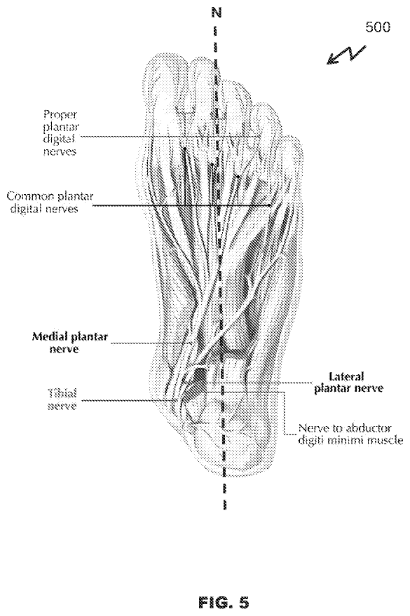

Referring now to FIG. 5, an illustration of a foot 500 shows a location of nerves within the foot 500 with the foot viewed from below. The foot 500 includes a longitudinal axis N shown in FIG. 5. The nerves of the foot include the tibial nerve, the lateral plantar nerve, the medial plantar nerve, and in the frontal regions of the foot 500, the common plantar digital nerves and proper plantar digital nerves. FIG. 5 also illustrates the nerve to the abductor digiti minimi muscle. As illustrated in FIG. 5, a path of each of the plantar nerves extends in a general longitudinal direction within the foot 500.

A relationship between a direction of a magnetic field and a direction of the induced electric fields is well known. That is, an electric field induced by a magnetic field is produced perpendicular to the direction of the magnetic field. Applicant employed this relationship to build an apparatus to deliver treatment with a time-varying magnetic field in desired direction relative to the plantar nerves when the treatment is applied to a treatment area of the subject.

That is, a magnetic field directed vertically through the foot from bottom to top will induce horizontal circular electric currents in the foot which are of minimal amplitude in the center of the foot, and maximal on the lateral and medial edges of the foot. As illustrated in FIG. 5, the plantar nerve has branches which run along the outside of the foot (specifically, branches leading to the 1st and 5th toes), and so these segments are in regions of the foot 500 where the induced electric fields could influence their behavior. The branches of the plantar nerve travelling longitudinally down the center of the front plantar surface, however, would be minimally influenced by any induced electric fields created by a magnetic field oriented perpendicular to the sole of the foot.

Applicant applied an understanding of the physiology of the foot with the physics concerning magnetic fields to build embodiments that optimize results from neuromodulation of the plantar nerves. In particular, embodiments described herein apply a magnetic field tangential to the plantar surface, directed from the medial to the lateral sides of the foot, to produce induced electric fields oriented in a line from the toes to the heel. That is, embodiments described herein deliver a treatment with a magnetic field oriented to produce induced electric fields in line with most branches of the plantar nerve in the front of the foot.

According to one embodiment, a two-coil apparatus utilizes two adjacent elliptical coils 10 cm on the long axis and 5 cm on the short axis. Each coil wound with 75 turns of #22 magnet wire, resulting in a coil of 750 microHenry. When series connected in an opposed fashion (so that the magnetic flux of each coil was in opposite directions to coils adjacent to it within the device) the magnetic flux in a foot placed over the coils is, therefore, primarily in the horizontal direction, maximizing the intensity of the induced electric fields along the length of the foot, in particular, along the branches of the plantar nerve running down the length of the foot.

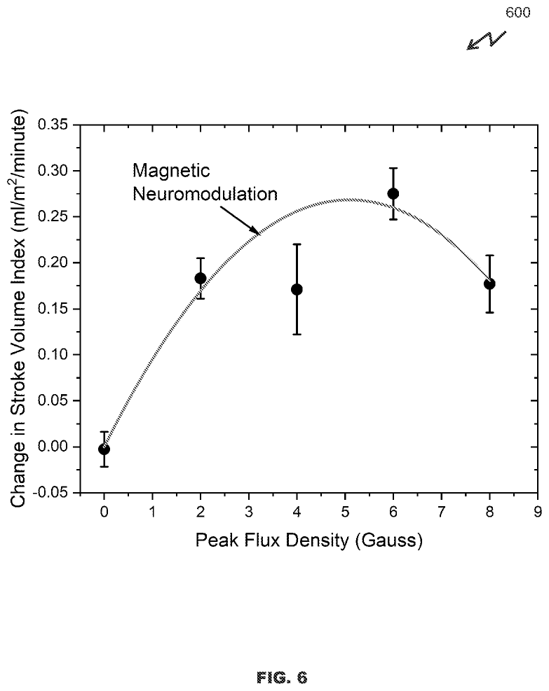

Referring now to FIG. 6, a plot 600 of change in stroke volume index versus peak flux density is illustrated in accordance with an embodiment in which the magnetic field is oriented horizontally relative to a surface on which a subject's foot is placed. That is, perpendicular to the longitudinal axis N of the foot 500 as illustrated, for example, in FIG. 5. FIG. 6 includes an x-axis of peak flux density in Gauss and a y-axis of the stroke volume index in milliliters per square meter per minute. Here too, a stroke volume index is used to normalize the results as determined for stroke volume across the varying body size of subjects. FIG. 6 illustrates the varied effectiveness of 4 Khz sinusoidal magnetic nerve modulation of the plantar nerves to achieve soleus activation with changes in flux density of the applied magnetic field using an embodiment having a two-coil arrangement to deliver a magnetic field exposure tangential to the foot. At each value of flux density tested, the range of values of change in stroke volume index were recorded.

At each of these values of flux density tested in this embodiment, the plot 600 includes a vertical line representing the standard deviation and a single data point representing the average value recorded at the selected flux density. Polynomial fitting of the data indicates a peak response around 5 Gauss, at a level of greater than 0.25 ml/m² per minute. This is more than twice the response observed with the vertical magnetic field arrangement, for example, as illustrated and described with reference to FIG. 4.

These experimental results demonstrate the superiority of a magnetic field orientated in a medial-lateral direction relative to a surface on which the foot is located as compared with results from the vertical field orientation. In particular, the horizontal field orientation delivers a 2× greater change in stroke volume as compared with a vertical field orientation. Also, the peak effective flux density is more distinct with the horizontal field orientation. According to these embodiments, application of a 4 KHz sinusoidal magnetic field produces a maximal response of cardiac output achieved at a flux density near 5 Gauss peak (0.5 mT peak). In addition, the maximally effective flux density is well below the exposure standards limit of 353 mT RMS (494 mT peak) for human limbs, and the even more strict limit of 0.87 mT peak standard for the head and torso. This confirms that these embodiments are inherently safe because they operate well below the IEEE exposure standards for any part of the body including those applicable to either the head or the torso, and recognizing that the IEEE standards include substantial safety factors. Reference here to the safe exposure levels established in this industry standard also highlights the significance of Applicant's findings because this standard would otherwise inform one of ordinary skill in the art that the flux density of the magnetic field included in this course of treatment is inconsequential when applied to a human subject. That is, the IEEE standard informs those of ordinary skill in that art that application of a magnetic field at this flux density does not result in any medical intervention and need not be tried.

The nature of soleus muscle operation also required that Applicant evaluate the duty cycle at which treatment is applied. In particular, the soleus muscles' function as the secondary hearts within the body results in an operation involving both a contraction phase—during which blood and lymphatic fluid are pumped out of the sinuses in the muscle and back to the heart; and a relaxation phase—during which the muscle fibers recover from fatigue, and the venous sinuses in the muscle refill with blood. Applicant recognized that a continuous activation of the muscle is ineffective as a stimulation strategy. For example, a continuous activation of the soleus muscles will result in the muscle cells becoming progressively more fatigued. In addition, the sinuses do not have an opportunity to refill if a treatment results in a continuous activation of the muscle. Conversely, brief activation of the soleus muscles repeated with long resting intervals also fails to have a significant long-term effect on venous return to the heart. Therefore, Applicant evaluated various embodiments to identify an optimal muscle activation time followed by an optimal muscle relaxation time. That is, Applicant evaluated different embodiments to identify an optimized duty-cycle for activation of the soleus muscles.

Applicant applied different treatment in various embodiments to isolate the optimal duty cycle. These included using a two-minute stimulus “on” period, and a magnetic field “off” time of various durations to achieve duty cycles of 20%, 33%, 50%, and 66%, as well as investigating the effect of continuous muscle activation (100% duty cycle referring to the stimulus being constantly on). Here, the term “on” refers to the portion of the treatment period with the magnetic field being active/on. The “on portion of the treatment period is followed by a period when the magnetic field is turned off. Applicant notes that a “duration” of treatment for any session determines the number of treatment periods. For example, if a treatment duration of 30 minutes is utilized, with 2 minutes of “on” times and 3 minutes of “off” times per treatment period, this results in a total of 6 five-minute treatment periods.

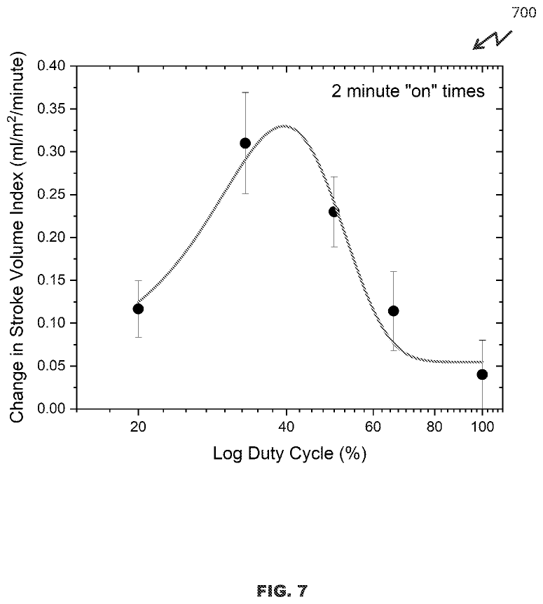

Referring now to FIG. 7, a plot 700 illustrating the effect of changes in a duty cycle of neuromodulation treatment on cardiac output is provided in accordance with one embodiment. FIG. 7 includes an x-axis of log duty cycle in percent and a y-axis of the change in stroke volume index in milliliters per square meter per minute. Again, a stroke volume index is used to normalize the results as determined for stroke volume across the varying body size of subjects. FIG. 7 illustrates the varied effectiveness of 4 Khz sinusoidal magnetic nerve modulation of the plantar nerves to achieve soleus activation with changes in duty cycle of the applied magnetic field using an embodiment that delivers a magnetic field exposure tangential to the foot. At each value of duty cycle tested, the range of values of change in stroke volume index were recorded. At each of the values of log duty cycle tested in this embodiment, the plot 700 includes a vertical line representing the standard deviation and a single data point representing the average value recorded at the selected duty cycle.

FIG. 7 illustrates cardiac hemodynamic response to variations in the duty cycle of the neuromodulation intervention for a constant two-minute neuromodulation period. According to these embodiments, a 20% duty cycle is associated with an 8 minute “relaxation” period for a treatment period of ten minutes. A 20% duty cycle is represented by the leftmost value plotted in FIG. 7. A continuous stimulus reflects a condition of no “relaxation” period, a 100% duty cycle. A 100% duty cycle is represented by the rightmost value plotted in FIG. 7.

Applicant finds that, consistent with expectations, a continuous application of the neuromodulation intervention (100% duty cycle) resulted in a very low effectiveness for the reasons described above. According to the embodiment illustrated in FIG. 7, the effectiveness of the stimulus applied using a 100% duty cycle is not statistically different from zero. According to the illustrated embodiment, this is consistent with the expectation that if no time is allowed for the soleus muscle to refill with blood and interstitial fluid, then there will be a minimal amount of fluid collected in the muscle which can be pumped out during the soleus muscle contraction phase. In addition, lack of a relaxation period precludes the muscle fibers from recovering, resulting in the build-up of fatigue.

Applicant also finds that a course of treatment including a very long “pause” (or “off” period), for example, a 20% duty cycle, also has low effectiveness. This is consistent with the expectation that once the sinuses of the soleus muscles have filled, further wait time is non-productive. Correspondingly, effectiveness of magnetic neuromodulation demonstrated a peak effectiveness between 20% and 100% duty cycle, when the neuromodulation duration was maintained at two minutes. Clinical testing performed by Applicant found that the greatest level of effectiveness was observed using a modulation pattern of approximately two minutes of “On” followed by approximately three minutes of “pause” (i.e. relaxation). According to the embodiment illustrated in FIG. 7, Gaussian curve fit to the data indicates that the optimal duty cycle is around approximately 40%.

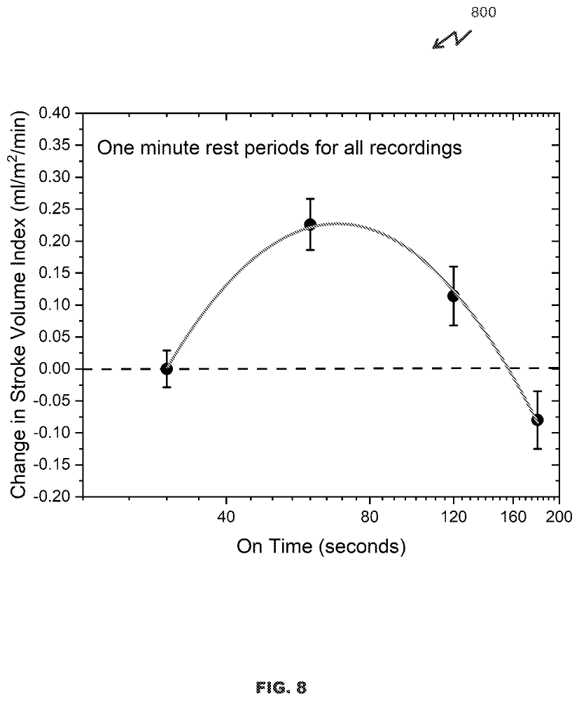

Applicant performed additional testing that varied the on-time with values other than two minutes to evaluate the impact of the length of the “on” period for treatment using neuromodulation to activate the soleus muscle. For example, Applicant performed this additional testing to find whether two minutes (120 seconds) may be too short a duration to effect complete contraction, or alternatively, that two minutes (120 seconds) may be a far longer duration than the soleus muscles need to complete a contraction. Referring now to FIG. 8, a plot 800 illustrating the effect of changes in on-time of neuromodulation treatment on cardiac output is provided in accordance with one embodiment. FIG. 8 includes an x-axis of on-time in seconds and a y-axis of the change in stroke volume index in milliliters per square meter per minute. Here again, a stroke volume index is used to normalize the results as determined for stroke volume across the varying body size of subjects. FIG. 8 illustrates results of testing performed at each of 30, 60, 120, and 180 seconds of neuromodulation while maintaining a pause period of one minute for all stimulation conditions. At each of these values of on-time, the plot 800 includes a vertical line representing the standard deviation and a single data point representing the average value recorded at the selected on-time.

The plot 800 illustrated in FIG. 8 provides a polynomial fitting of the data. This illustrates that neuromodulation durations in the range of 50-90 seconds are superior for activating the soleus muscles, with the optimal time of about 70 seconds. While identifying the optimum on-time, Applicant also finds that results of this testing indicate that the peak response is not as great as that observed in FIG. 7. This indicates that the one minute “relaxation” time utilized in this study is insufficient to permit complete refilling of the muscle, and a larger response is likely with a longer relaxation time, specifically, a relaxation time consistent with a 40% duty cycle.

Additional studies performed by Applicant indicate that the optimal neuromodulation duration for activating the soleus muscles is in the range of 50-90 seconds with a duty cycle of about 40%. This appears to provide the appropriate time for both muscle sinus refilling and recovery from fatigue. According to one embodiment, a 70 second neuromodulation duration, with a relaxation time of about 100 seconds are appropriate as an optimally designed neuromodulation pattern for the plantar nerve.

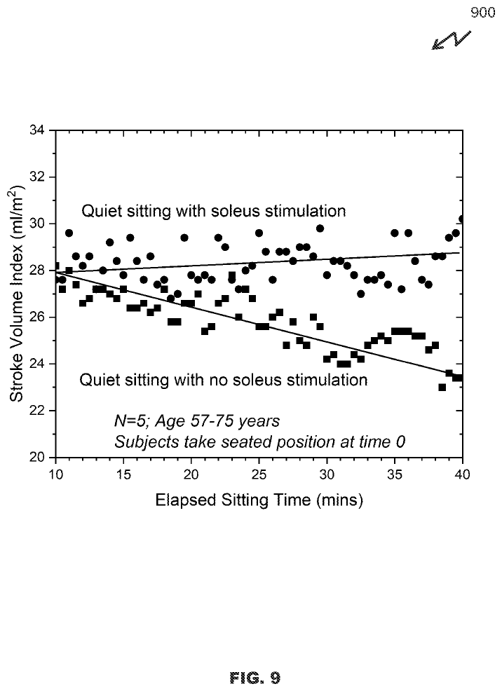

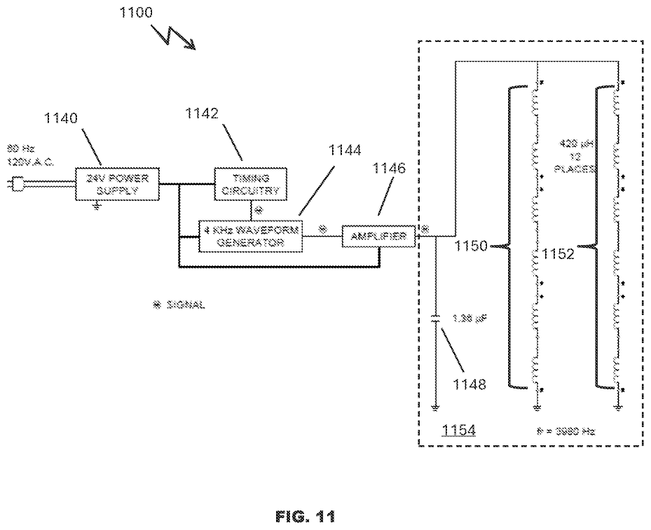

As described further below, Applicant has also assessed the effectiveness of various coil configurations, coil orientations and quantity of coils to improve the performance and ease of use of a neuro modulation treatment device to activate the soleus muscle for treatment of reduced cardiac output. Referring now to FIG. 9, a plot 900 illustrates a summary graphic of results of neuromodulation of the plantar nerve in terms of its effects on cardiac output determined by stroke volume index in individuals seated quietly. The plot 900 also reflects results using an embodiment of the treatment apparatus illustrated and described herein with reference to FIGS. 11 and 12. Further, these results also reflect results with the subjects being treated with their shoes on and the treatment apparatus operated to deliver a magnetic flux of 5 Gauss peak (1 cm above the treatment surface) at 4 KHz, using a 50% duty cycle signal with a neuromodulation duration of one minute.

The plot 900 includes an x-axis of time in minutes and a y-axis of the stroke volume index in milliliters per square meter. In FIG. 9, the measured time period is from 10-40 minutes following a start time at which the subject is seated. The set of measurements taken for a control (i.e., data recorded without neuromodulation being applied to the subject) is illustrated in square data points representing the result values. Circular data points represent result values when neuro-modulation treatment is applied to stimulate the soleus muscle. A linear curve fit representative of the rate of change in stroke volume is represented by a straight line associated with each of the sets of data, respectively.

The control data points in FIG. 9 illustrate a continuous decrease in stroke volume index during a period of quiet sitting in the absence of neuromodulation. This is generally represented by the negative slope of the curve fit for these data points. The overall decrease in stroke volume is close to 20% during the thirty-minute measurement window for subjects evaluated in this study. Conversely, with neuromodulation applied, these same subjects achieve an increase in stroke volume index during the thirty-minute measurement window. This is generally represented by the positive slope of the curve fit for these data points. These results demonstrate that neuromodulation as described herein prevents fluid pooling. This is evidenced by the increase in stroke volume over time which is consistent with enhanced venous return to the heart. These results are provided in combination with the reversal of fluid pooling as previously illustrated and described with reference to FIGS. 2 and 3.

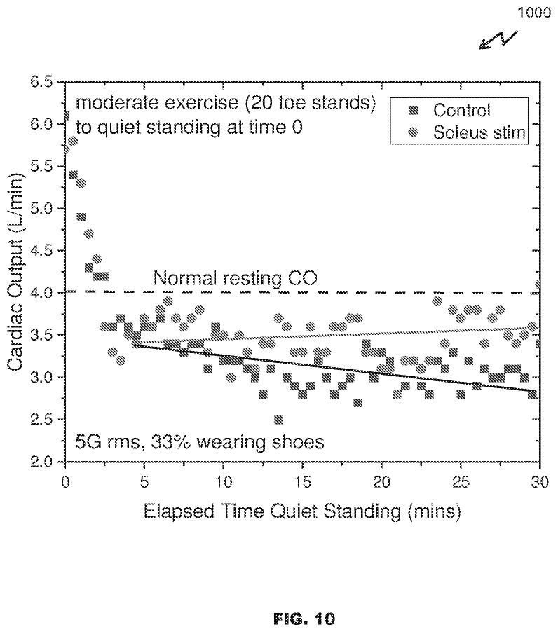

FIG. 10 illustrates a plot 1000 that provides a summary graphic of results of neuromodulation of the plantar nerve in terms of its effects on cardiac output determined by stroke volume and heart rate in individuals standing quietly following a period of moderate intensity exercise (in this case, a total of 20 toes stands). The plot 1000 also reflects results using an embodiment of the treatment apparatus illustrated and described herein with reference to FIGS. 11 and 12 with the subject being treated with their shoes on and the treatment apparatus operated to deliver a magnetic flux of 5 Gauss peak (1 cm above the treatment surface) at 4 KHz, using a 50% duty cycle signal with a neuromodulation duration of one minute. The plot 1000 includes an x-axis of time in minutes and a y-axis of cardiac output in liters per minute. In FIG. 10, the measured time period is from 0-30 minutes following a start time at which the subject ends their moderate intensity exercise. Here too, the set of measurements taken for a control (i.e., data recorded without neuromodulation being applied to a patient) is illustrated in square data points representing the result values. The plot 1000 employs circular data points to represent result values when neuro-modulation treatment is applied to stimulate the soleus muscle. A curve fit representative of the rate of change in cardiac output is represented by a straight line associated with each of the sets of data, respectively. Normal resting cardiac output is represented by a dashed horizontal line at approximately 4 liters per minute.

The control data points in FIG. 10 illustrate an immediate and rapid decrease in cardiac output within the first 2-3 minutes of quiet standing following a period of moderate activity and in the absence of neuromodulation. Thereafter, cardiac output continues to steadily decrease for the remainder of the thirty-minute period represented in the plot 1000. This is generally represented by the negative slope of the curve fit for these data points. In greater detail, the test protocol illustrated that resting (supine rest) cardiac output is about 4-5 liters per minute. Twenty toe stands are seen to increase cardiac output by about 50%, to approximately 6 liters per minute. The change in cardiac output in response to the activity demonstrates that the cardiovascular system is healthy, and that with increased venous return, the subject's heart can increase its output. That is, the subject is not suffering from heart failure. When the activity is stopped and the subject remains standing quietly, cardiac output is seen to decline to about 3.5 liters per minute. In the absence of any plantar nerve modulation, cardiac output continues to decline another 15% over the remainder of the 30-minute period. This is generally represented by the negative slope of the curve fit associated with the plot of the control data points.

Conversely, with plantar nerve modulation applied during quiet standing, cardiac output remains constant at about 3.5 liters per minute for the next 30 minutes, indicating that in the standing position as well as the seated position, soleus muscle activation helps to maintain cardiac output while performing relatively sedentary activities. This is generally represented by the slight positive slope of the curve fit for these data points. These results demonstrate that a magnetic neuromodulation treatment applied to plantar nerve to activate the soleus muscle serves to maintain cardiac output over time. As a result, a magnetic neuromodulation of the plantar nerve is effective in preventing fluid pooling when an individual is in a quiet standing position. Thus, in various embodiments, the treatment protocols applying magnetic neuromodulation of the plantar nerve provide opportunities for individuals who perform standing tasks. This can be particularly effective for individuals who must stand for long periods.

Referring now to FIG. 11, a circuit diagram of circuitry 1100 included in an apparatus to provide a medical intervention for a user with reduced cardiac output is illustrated in accordance with one embodiment. In various embodiments, the circuitry 1100 is included in a medical device that generates a time varying magnetic field having a frequency and an intensity that triggers a response that activates a skeletal muscle to increase a venous return of blood and a lymphatic return of interstitial fluid to improve a cardiac output of the user. In some embodiments, the medical device is designed to provide treatment to the plantar region of a subject's foot to trigger a response that activates the soleus muscle to increase a venous return of blood and a lymphatic return of interstitial fluid to improve a cardiac output of the user.

According to the illustrated embodiment, the circuitry 1100 includes a power supply 1140, a timing circuit 1142, a waveform generator 1144 and an amplifier 1146. In various embodiments, the circuitry 1100 also includes a capacitor 1148, a first plurality of inductors 1150 and a second plurality of inductors 1152. According to the illustrated embodiment, power for the circuitry is supplied by connection to a commonly available AC power source, for example, 120 Volt, 60 Hz AC power in the U.S. and 230 Volt, 50 Hz AC power in Europe. Further, in the illustrated embodiment, the input AC voltage is reduced to 24 Volts DC by the power supply 1140. However, different output voltages can be employed depending on the embodiment. According to other embodiments, a connection to an external AC power source is not required. Instead, in these embodiments, the apparatus includes a battery power source and the circuitry 1100 includes power conversion circuitry to utilize DC power supplied by a battery power source or an integral DC generator.

In the illustrated embodiment, the output of the power supply 1140 is supplied to each of the timing circuit 1142, the waveform generator 1144 and the amplifier 1146. The output of the timing circuit 1142 is connected to an input of the waveform generator 1144. The output of the waveform generator 1144 is connected to the input of the amplifier 1146. According to the illustrated embodiment, the capacitor 1148 is connected between the output of the amplifier 1146 and ground. Each of the first plurality of inductors 1150 and the second plurality of inductors 1152, respectively, is also connected between the output of the amplifier 1146 and ground.

According to the illustrated embodiment, the first plurality of inductors 1150 includes six inductors connected in series between the output of the amplifier 1146 and ground. Similarly, the second plurality of inductors 1152 includes six inductors connected in series between the output of the amplifier 1146 and ground. Each inductor included in the plurality of inductors 1150, 1152 is illustrated with an associated marking indicating a polarity of the inductor, respectively. The inductors in each of the respective plurality of inductors 1150, 1152 are wired in series in alternating north and south field orientations. According to some embodiments, the apparatus is a medical treatment device configured to simultaneously apply treatment to both feet. In these embodiments, the first plurality of inductors 1150 are located within the device to deliver a time-varying magnetic field to a first of the subject's feet (for example, the left foot) while the second plurality of inductors 1152 are located within the device to deliver a time-varying magnetic field to a second of the subject's feet (for example, the right foot).

While the illustrated embodiment includes a total of twelve inductors, different quantities of inductors can be employed and placed in different combinations depending on the embodiment provided that the inductors are physically located in a position and orientation to deliver a time varying magnetic field to a selected treatment area for neuromodulation of a selected peripheral nerve to trigger a response that activates a skeletal muscle to increase a venous return of blood and a lymphatic return of interstitial fluid to improve a cardiac output of the user. As described in greater detail below, Applicant finds that the location, quantity and orientation of inductors in the apparatus can be adjusted to provide more effective treatment depending on several considerations. In summary, a quantity of inductors to be employed is determined, at least in part, by the physical dimensions of the inductor, the size of the treatment area, and the distance separating treatment areas where treatment is applied to multiple treatment areas simultaneously with a single apparatus.

In various embodiments, the capacitance value of the capacitor 1148 and the inductance value of the inductors 1150, 1152, respectively, are selected to provide a resonant circuit 1154 at a selected frequency. The output of the amplifier 1146 is connected to the resonant circuit 1154 formed by the combination of the capacitor 1148, the first plurality of inductors 1150 and the second plurality of inductors 1152. According to the illustrated embodiment, a combined total of twelve inductors are employed each having an inductance of 420 microHenry. The pluralities of inductors 1150, 1152, respectively, are connected in parallel with the capacitor 1148 having a capacitance of 1.36 microFarads. These values provide the resonant circuit 1154 with a resonant frequency of 3980 Hz, or approximately 4 KHz. Thus, the values are selected to provide treatment at the frequency found to provide the greatest decrease in vascular resistance as shown and described with reference to FIGS. 2 and 3. In other embodiments, the resonant circuit 1154 can be constructed and arranged to provide a different resonant frequency, for example, to provide the apparatus with an output frequency of the time-varying magnetic field at a different frequency tailored to a particular course of treatment.

In operation, the circuitry 1100 receives the external AC power at the input to the power supply 1140. The power supply 1140 provides a 24 Volt DC output that is provided to the timing circuitry 1142. The timing circuitry 1142 operates in a single treatment period by turning on and off to meet the duty cycle requirements of the course of treatment. When the timing circuitry is on, a trigger signal is delivered to an input of the waveform generator 1146. The waveform generator 1146 operates to provide a sinusoidal output that is provided to the amplifier 1144 which provides current to drive the resonant circuit 1154. When driven in this fashion, each respective inductor included in the plurality of inductors 1150 and 1152 generates a time varying magnetic field at the desired frequency, here 4 KHz. Provided the inductors are positioned correctly relative to the treatment area, this time varying magnetic field triggers the response that activates a skeletal muscle to increase a venous return of blood and a lymphatic return of interstitial fluid to improve a cardiac output of the user. When the timing circuitry is off, an output is not provided to the amplifier 1144. During this portion of the treatment period a time varying magnetic field is not produced by the circuitry 1100. As described herein, a single treatment period includes a combination of one or more on-period(s) and one or more off-periods(s) to provide the combination of duty cycle and resting period length tailored to deliver an optimized treatment. That is, a treatment intended to improve cardiac output most effectively.

According to one embodiment, the circuitry 1100 includes twelve inductors and operates at with a 70V peak drive voltage, and 0.74 amps resulting in 1.15 W of power consumption when “on”. According to the illustrated embodiment, the circuitry operates with an average power consumption of 0.58 W for 50% duty cycle operation. As is apparent here and as found by Applicant in other multi-coil embodiments, the circuitry provides low power consumption when “on” and minimal power consumption when off. Further, the embodiments of the circuitry 1100 illustrated and described herein include a resonant circuit that assists in significantly reducing power consumption. However, a resonant circuit is not necessary and some embodiments can include circuitry that does not include the resonant circuit.

Referring now to FIG. 12, an apparatus 1200 providing a medical treatment by generating a time varying magnetic field having a frequency and an intensity that triggers a response that activates the soleus muscles to increase a venous return of blood and a lymphatic return of interstitial fluid to improve a cardiac output of the user. According to the illustrated embodiment, the apparatus 1200 includes a first plurality of inductors 1250, a second plurality of inductors 1252, a housing 1256 including an interior 1257 and a handle 1258. Further, in some embodiments, the apparatus 1200 includes the circuitry 1100 (partially illustrated here by the inductors 1250, 1252 shown in phantom within the housing 1256) with a connection to an external power source. The interior 1257 includes one or more hollow cavities in which elements of the circuit 1100 illustrated and described with reference to FIG. 11 can be located. Where an integral battery power supply is used, the battery can be in the interior 1257. In the illustrated embodiment, the apparatus 1200 includes the first plurality of inductors 1250 located in the left half of the interior 1257 and the second plurality of inductors 1252 located in the right half of the interior 1257. In this embodiment, each of both the first plurality of inductors 1250 and the second plurality of inductors 1252 included a total of six inductors, respectively. Each of the respective inductors includes a coil 1260 of magnetic wire surrounding an air core 1262.

Applicant finds that the physical arrangement of the inductors within the apparatus 1200 is important for a delivery of the most effective treatment. In particular, Applicant finds that the inductors included in the plurality of inductors 1250, 1252, respectively, should be arranged in a linear array with alternating north and south poles to deliver the desired time varying (sinusoidal) magnetic field. Accordingly, the inductors included in the first plurality of inductors 1250 are located within the interior 1257 in a first linear array with the inductors having alternating north and south poles. Similarly, the inductors included in the second plurality of inductors 1252 are located within the interior 1257 in a second linear array with the inductors having alternating north and south poles. This arrangement positions the inductors in the two separate arrays such that a polarity of each of the plurality of electrical coils, respectively, is opposite a polarity of an electrical coil included in the plurality of electrical coils located adjacent the respective coil within the interior of the housing. Further, the two linear arrays are arranged within the cavity 1257 such that the leftmost array including the first plurality of inductors 1250 is positioned for treatment applied to the frontal plantar region of a subject's left foot while the rightmost array including the second plurality of inductors 1252 is positioned for treatment applied to the frontal plantar region of a subject's right foot.

According to various embodiments, each of the inductors 1250, 1252 is an air-core coil constructed with 65 turns of #24 magnetic wire to provide an inductance of 420 microHenry, respectively. Coils with different electrical characteristics or composed of different materials (for example, iron core coils) can also be employed in various embodiments, preferably employed with a capacitor having a capacitance such that the combination forms a resonant circuit having a desired resonant frequency for treatment to reduce power consumption. In the illustrated embodiment, this construction provides an inductor that is 2.5 cm wide and 8.5 long. Coils with different dimensions can also be employed in various embodiments provided they are sized and arranged within the housing 1256 to deliver the time varying sinusoidal magnetic field to a sufficiently large percentage of the treatment area, here the frontal plantar region of the subject's foot. However, Applicant finds that relatively narrow inductors as provided in the illustrated embodiment ensure that, for even very narrow feet, wherever the individual placed their feet, they would be covering at least two coils. This, in turn, ensured that a large portion of the frontal plantar surface would be exposed to a horizontal magnetic field independent of the precise location of the subject's foot on the treatment surface of the device.

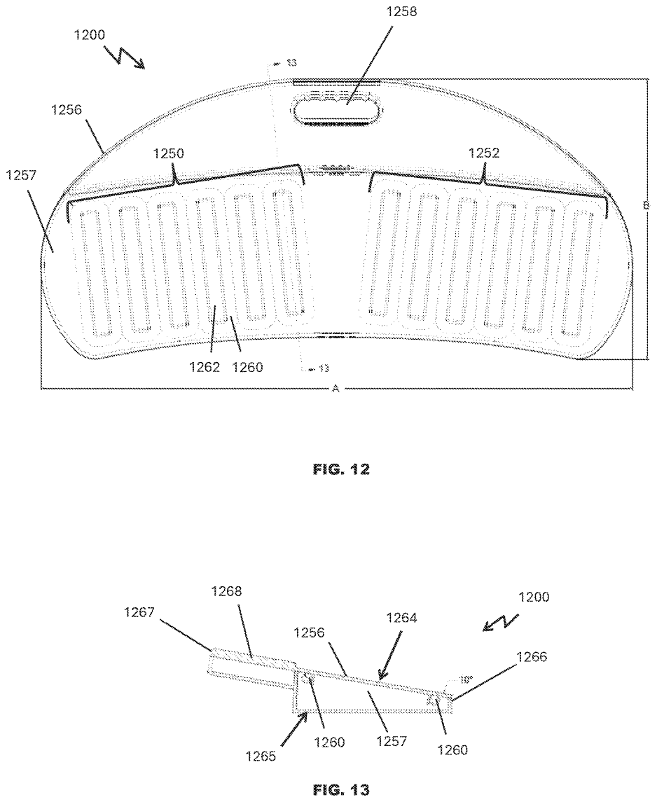

FIG. 13 illustrates a cross-sectional view of the apparatus 1200 of FIG. 12. This view shows the housing 1256 including the interior 1257, a top surface 1264, a bottom surface 1265, a proximate end 1266 and a distal end 1267. This view also provides a cross-sectional view of elements of the coil 1260. The distal end 1267 also includes a raised region 1268. According to the illustrated embodiment, a first region of the top surface 1264 located over much of the top surface including that closest to the proximate end 1266 is sized and configured to receive the frontal plantar region of the foot. A second region of the top surface 1264 closer to the distal end 1267 is sized and configured to receive the toes, for example, on the raised region 1268.

In general, the form factor of the apparatus 1200 is configured to provide a portable treatment device that simultaneously delivers a sinusoidal time-varying magnetic field tangentially to the sole of both feet of a patient. FIG. 12 includes reference to a width A and a depth B of the apparatus 1200. According to one embodiment, the apparatus 1200 has a dimension A of twenty-one inches and a dimension B of thirteen inches. The handle 1258 is located at the distal end 1267 of the apparatus 1200. The flat bottom surface 1265 allows the apparatus 1200 to be placed on a flat surface such as the floor. In this orientation, a seated user can place their left foot on the region of the top surface 1264 located directly above the first plurality of inductors 1250 and their right foot above the second plurality of inductors 1252. Applicant finds that by angling the treatment surface slightly upward comfort can be substantially improved for the subject's feet. For clarity, this is reflected in the 10-degree angle between the top surface 1264 and the bottom surface 1265 as illustrated in FIG. 13. Further, Applicant's clinical testing finds that effective treatment can be provided with a subject in bare feet, stocking feet or when wearing shoes. FIG. 10 illustrates one such example. Applicant recognized the contactless nature of treatment using a magnetic field provides more flexibility in this regard than conventional electrical stimulation devices. In particular, the time varying magnetic field delivered by the embodiments described herein penetrates all non-magnetic materials. Thus, treatment apparatus can be configured for use with one or more pieces of attire (including shoes) located between a treatment surface of the apparatus (for example, the top surface 1264) and the subject's skin in the vicinity of the treatment area of the subject.

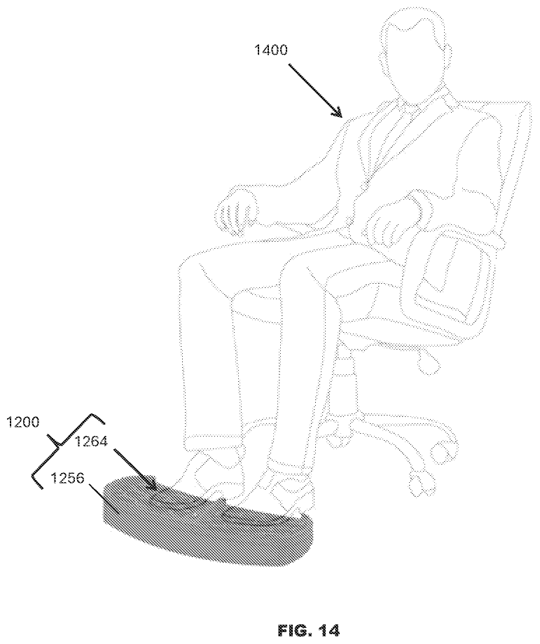

Referring now to FIG. 14, a subject 1400 is shown employing the apparatus 1200 while seated in a desk chair while wearing shoes. Here, an exterior of the housing 1256 is shown including the top surface 1264. The subject 1400 has placed the soles of their shoes, in the region of the balls of the feet, on the top surface. In an embodiment of the device as illustrated and described with reference to FIGS. 11-13, the subject 1400 can initiate treatment by turning the circuit on using an on/off switch (not illustrated) accessible at an exterior of the housing 1256. Alternatively, the device could include a sensing system to detect the presence of a foot and automatically turn on.

While the embodiments illustrated and described herein include a treatment surface of the apparatus (for example, the top surface 1264 of the apparatus 1200) that is placed adjacent a treatment surface of the subject (for example, the sole of the foot and, in particular, the ball of the foot), other relative positions can be employed in further embodiments. In accordance with some embodiments, a treatment apparatus is configured such that the subject's treatment area is located between one or more inductors where the inductors are included in circuitry generally operated as shown and described with reference to FIG. 11 include herein. For example, where the treatment area is the frontal plantar region of the subject's foot, a treatment apparatus can be configured such that the subject's foot is located between two elements that include inductors. According to these embodiments, the foot is “sandwiched” between inductors located on either side of the foot for application of magnetic field to provide neuromodulation of the plantar nerve.

Applicant also finds that that the treatment described in various embodiments herein can be applied to the plantar region on each foot independent of the other. That is, an effective treatment protocol can apply the time varying magnetic field to a treatment area on a first of the subject's feet during a first period in which the other of the subject's feet is not receiving treatment. During a second period that follows the first, the time varying magnetic field is applied to a treatment area on the second of the subject's feet. Such an approach may be used, for example, with a smaller and even more portable treatment device that is sized such that only a single foot can be placed on the treatment surface of the apparatus. Applicant recognizes that this approach also triggers a response that activates a skeletal muscle to increase a venous return of blood and a lymphatic return of interstitial fluid to improve a cardiac output of the user.

While the embodiments illustrated and described herein include multiple inductors, according to some embodiments, only a single inductor is employed for application of magnetic field to provide neuromodulation to a subject. According to these embodiments, the inductor is sized and arranged to provide a physical dimension appropriate to deliver the time varying magnetic field to a treatment area of the patient to trigger a response that activates a skeletal muscle to increase a venous return of blood and a lymphatic return of interstitial fluid to improve a cardiac output of the user.

Reduced cardiac output is known to contribute to a variety of related conditions including but not limited to: heart failure; drug resistant hypertension, cognitive impairment; slow/delayed wound healing; varicose veins; deep vein thrombosis; macular degeneration; age related hearing loss; osteoporosis; and sleep apnea. Each of the preceding conditions is an indicator that a subject is suffering from a reduced cardiac output. Accordingly, Applicant has evaluated various treatments using the apparatus and methods described herein to determine their impact in treating these conditions. In general, Applicant finds that the treatment should be performed for at a minimum of one hour per day to improve performance of the soleus muscle during periods outside of the treatment sessions. A more rapid rejuvenation of the soleus muscle is found to occur with 2-3 hours of treatment daily. A study by Applicant of subjects suffering from osteoporosis, found that 2.5 hours/day was sufficient to stop, or reverse, bone loss over the course of a one year. A cognitive aging study by Applicant found that 1-2 hours per day was sufficient to reverse cognitive impairment within six months. Further, Applicant finds that the ability of the treatments described herein to decrease vascular resistance (see for example, FIGS. 2, 3 and their associated description) results in an opportunity to apply a time-varying magnetic field to the plantar region to lower blood pressure in hypertensive individuals in a non-pharmacologic manner.

While Applicant's clinical studies find that soleus muscle recovery time generally requires about three months using the treatment for an hour a day, a sustained course of treatment should be employed to prevent atrophy of the soleus muscle over time that would otherwise occur if treatment is stopped. This approach provides subjects treated for low resting cardiac output with lasting improvements in cardiac output that can address specific medical conditions while also improving the subject's overall quality of life.

While the above-described embodiments refer to magnetic nerve modulation in the foot, the apparatus, system and method illustrated and described herein can be employed on other elements of human anatomy in various embodiments.

Having thus described several aspects of at least one embodiment of this invention, it is to be appreciated that various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

1. An apparatus configured for a non-invasive medical intervention for a user with reduced cardiac output, the medical device comprising:
at least one electrical coil; and
power control circuitry configured to provide an electrical output supplied to the at least one electrical coil at a voltage and frequency selected to drive current through the at least one electrical coil to generate a time varying magnetic field having a frequency and an intensity that triggers a response that activates a skeletal muscle to increase a venous return of blood and a lymphatic return of interstitial fluid to improve a resting cardiac output of the user,
wherein the frequency of the time varying magnetic field includes a range of 2-16 KHz, and
wherein a flux density of the time varying magnetic field includes a range of 2-8 Gauss peak.

2. (canceled)

3. The apparatus of claim 1, wherein the frequency of the time varying magnetic field is selected because the non-invasive medical intervention at the selected frequency results in both stopping fluid pooling in the user and reversing existing fluid pooling in the user.

4. The apparatus of claim 1, wherein the selected frequency of the time varying magnetic field is substantially equal to 4 KHz.

5. The apparatus of claim 7, further comprising timing circuitry configured to control the output of the power control circuitry to generate the time varying magnetic field at a duty cycle of 30%-50%.

6. The apparatus of claim 5, wherein the duty cycle of the time varying magnetic field is substantially equal to 40%.

7. The apparatus of claim 1, wherein the timing circuitry is configured to control the output of the power control circuitry to periodically generate the time varying magnetic field for a period of 50-90 seconds.

8. The apparatus of claim 7, wherein the period is substantially equal to 70 seconds.

9. The apparatus of claim 1, further comprising a housing having an interior configured to house the at least one electrical coil, and an exterior surface configured to place against a treatment area of a user with the treatment area located in a predetermined orientation relative to the exterior surface,
wherein the treatment area is focused on a frontal plantar region including a plantar nerve in a foot of the user, and
wherein the predetermined orientation results in the time varying magnetic field being oriented in a direction perpendicular to the plantar nerve.

10. The apparatus of claim 9, wherein the at least one electrical coil includes a plurality of electrical coils, and
wherein a polarity of each of the plurality of electrical coils, respectively, is opposite a polarity of an electrical coil included in the plurality of electrical coils located adjacent the respective coil within the interior of the housing.

11.-14. (canceled)

15. The apparatus of claim 1 for use in a method of treating a medical condition in a human subject where the method includes:
positioning the at least one electrical coil adjacent a treatment area of the human subject, the treatment area including a targeted nerve;
orienting the at least one electrical coil to locate the time varying magnetic field produced with the at least one electrical coil in a direction perpendicular to the targeted nerve; and
operating the apparatus to generate the time varying magnetic field at the frequency and the intensity that triggers the response that activates the skeletal muscle to increase the venous return of blood and the lymphatic return of interstitial fluid to improve the resting cardiac output of the human subject.

16. The apparatus of claim 1 for use in a method of treating a medical condition in a human subject where the method includes:
applying the time varying magnetic field to a treatment area located in a foot of the human subject; and
selecting each of a frequency of the time varying magnetic field, a flux density of the time varying magnetic field, a period for application of the time varying magnetic field, and a duty cycle for the application of the time varying magnetic field to trigger a response that activates a soleus muscle to increase a venous return of blood and a lymphatic return of blood to improve a resting cardiac output of the human subject.

17. A method of improving cardiac output with an apparatus including at least one electrical coil, the method comprising:
positioning the at least one electrical coil adjacent a treatment area of a user, the treatment area focused on a frontal plantar region of a foot of the user, the treatment area including a plantar nerve located in the treatment area;
orienting the at least one electrical coil to locate a time varying magnetic field produced with the at least one electrical coil in a direction perpendicular to the plantar nerve; and
operating the apparatus to generate the time varying magnetic field at a frequency and an intensity to trigger a response that activates a soleus muscle to increase a venous return of blood and a lymphatic return of interstitial fluid to improve a resting cardiac output of the user.

18. The method of claim 17, further comprising selecting a frequency value of the time varying magnetic field from within a range of 2-16 KHz, the frequency value selected because a treatment at the selected frequency results in both stopping fluid pooling in the user and reversing existing fluid pooling in the user.

19. The method of claim 17, further comprising:
generating the time varying magnetic field with the frequency in a range of 2-16 KHz; and
generating the time varying magnetic field with a flux density of 2-8 Gauss peak.

20. The method of claim 17, further comprising generating the time varying magnetic field at a duty cycle of 30%-50%.

21. The method of claim 17, further comprising periodically generating the time varying magnetic field for a period of 50-90 seconds.

22. The method of claim 17, wherein the act of positioning includes an act of positioning the at least one electrical coil adjacent to a surface of the skin adjacent to the treatment area.

23. A method of improving cardiac output with an apparatus including at least one electrical coil, the method comprising:
a) positioning the at least one electrical coil adjacent a surface of a treatment area of a user, the treatment area including a targeted nerve;
b) orienting the at least one electrical coil to locate a time varying magnetic field produced with the at least one electrical coil in a direction perpendicular to the targeted nerve; and
c) operating the apparatus to generate the time varying magnetic field at a frequency in a range of 2-16 KHz and a flux density of 2-8 Gauss peak to trigger a response that activates a skeletal muscle to increase a venous return of blood and a lymphatic return of interstitial fluid to improve a resting cardiac output of the user.

24. The method of claim 23, further comprising generating the time varying magnetic field during a period of 50-90 seconds at a duty cycle of 30%-50%.

25. A method of treating an individual for a medical condition resulting, at least in part, from a reduced cardiac output, the method comprising:
applying a time varying magnetic field to a treatment area located in the individual's foot, the treatment area focused on a frontal plantar region including a plantar nerve in the individual's foot, the time varying magnetic field oriented in a direction perpendicular to the plantar nerve; and
selecting each of a frequency of the time varying magnetic field, a flux density of the time varying magnetic field, a period for application of the time varying magnetic field, and a duty cycle for the application of the time varying magnetic field to trigger a response that activates a soleus muscle to increase a venous return of blood and a lymphatic return of blood to improve a resting cardiac output of the user.

26. The method of claim 25, further comprising:
positioning at least one electrical coil adjacent a surface of the individual's foot; and
orienting the at least one electrical coil to produce the time varying magnetic field in the direction perpendicular to the plantar nerve.

27. The method of claim 26, wherein a stroke volume index with the subject at rest returns to a value at which individuals that do not suffer from a reduced cardiac output experience.

28. The method of claim 26, further comprising applying the time varying magnetic field at a selected duty cycle for at least one hour per day.

29. The method of claim 28, wherein the subject suffers from a medical condition including any of the following: heart failure; drug resistant hypertension; cognitive impairment; delayed wound healing; varicose veins, deep vein thrombosis, macular degeneration; age related hearing loss; osteoporosis; and sleep apnea, and wherein the method further comprises an improvement in the medical condition.

30. The method of claim 29, wherein the medical condition is a cognitive impairment, and wherein the method includes improving a cognitive performance of the subject following an application of the time varying magnetic field at the selected duty cycle for one to two hours per day for six consecutive months.

31. The method of claim 29, wherein the medical condition is osteoporosis, and wherein the method includes stopping a bone loss in the subject following an application of the time varying magnetic field at the selected duty cycle for at least two hours per day for up to twelve consecutive months.

32. A method of increasing brain blood flow with the method of claim 25.

33. A method of normalizing blood pressure in a patient diagnosed with chronically low blood pressure with the method of claim 25.
